(19) United States
(12) Patent Application Publication
(10) Pub. No.: US 2023/0273404 A1
(43) Pub. Date: Aug. 31, 2023

(54) OBJECTIVE OPTICAL SYSTEM, IMAGE PICKUP APPARATUS, AND ENDOSCOPE

(71) Applicant: OLYMPUS MEDICAL SYSTEMS CORP., Tokyo (JP)

(72) Inventor: Yoshihiro KAWAKAMI, Tokyo (JP)

(73) Assignee: OLYMPUS MEDICAL SYSTEMS CORP., Tokyo (JP)

(21) Appl. No.: 18/314,259

(22) Filed: May 9, 2023

Related U.S. Application Data
(63) Continuation of application No. PCT/JP2021/021919, filed on Jun. 9, 2021.

(51) Int. Cl. G02B 13/00

(52) U.S. Cl. CPC G02B 13/0015; A61B 1/00096

(57) ABSTRACT

An objective optical system includes a front group FG, an aperture stop S, and a rear group RG in order from an object side. The front group FG includes a first lens L1 having a negative refractive power with a concave surface facing an image side, a meniscus-shaped second lens L2 having a negative refractive power with a convex surface facing the image side, and a third lens L3 having a positive refractive power. The rear group RG includes a fourth lens L4 having a positive refractive power and a fifth lens L5 having a positive refractive power. The fifth lens L5 is a cemented lens. The following Conditional Expression (1) is satisfied: 2.3<f3/f5<20 (1), where f3 is a focal length of the third lens L3, and f5 is a focal length of the fifth lens L5.

### CROSS-REFERENCE TO RELATED APPLICATION

The present application is a continuation application of PCT/JP2021/021919 filed on Jun. 9, 2021; the entire contents of which are incorporated herein by reference.

### TECHNICAL FIELD

### Background

The present disclosure relates to an objective optical system, an image pickup apparatus, and an endoscope.

### Description of the Related Art

In electronic endoscopes in the medical field, skills related to treatment and diagnosis have been advanced. An electronic endoscope is inserted into a living body to find or detect more minute lesions. Electronic endoscopes for 2D observation provide planar (two-dimensional) observation. Electronic endoscopes for 3D observation provide stereoscopic (three-dimensional) observation. In both cases of 2D observation and 3D observation, accurate focus adjustment to the image pickup surface of an imager is essential at the time of manufacturing of an objective optical system. Furthermore, an endoscope objective optical system observes a lesion in close proximity during treatment and diagnosis. Thus, if the distance between the respective optical axes of the objective optical systems for the left eye and the right eye, that is, the baseline length is large, the parallax between an image for the left eye and an image for the right eye is also large. For this reason, the observer tends to have eye strain during 3D observation. It is therefore necessary to set a short baseline length in the endoscope objective optical system for 3D observation. Moreover, stereoscopic viewing tends to be difficult if the resulting image position is displaced from an image position appropriate for 3D observation due to decentration of the objective optical systems for the left eye and the right eye.

As a conventional objective optical system, for example, Japanese Unexamined Patent Application Publication No. 2018-159853 discloses a five-group configuration having refractive power arrangement of negative, negative, positive, aperture, positive, and cemented (positive-negative) in order from the object side.

### SUMMARY

An objective optical system according to at least some embodiments of the present disclosure includes: a front group; an aperture stop; and a rear group in order from an object side, wherein
the front group includes a first lens having a negative refractive power with a concave surface facing an image side, a meniscus-shaped second lens having a negative refractive power with a convex surface facing the image side, and a third lens having a positive refractive power,
the rear group includes a fourth lens having a positive refractive power and a fifth lens having a positive refractive power,
the fifth lens is a cemented lens, and
the following Conditional Expression (1) is satisfied:

2.3<f3/f5<20 (1)
where
f3 is a focal length of the third lens, and
f5 is a focal length of the fifth lens.

Furthermore, an image pickup apparatus according to at least some embodiments of the present disclosure includes: an objective optical system; and an imager disposed on an image plane, wherein
the imager has an image pickup surface and converts an image formed on the image pickup surface by the objective optical system into an electrical signal, and
the objective optical system is the objective optical system described above.

An endoscope according to at least some embodiments of the present disclosure includes the image pickup apparatus described above.

### BRIEF DESCRIPTION OF THE DRAWINGS

FIG. 1 is a lens cross-sectional view of an objective optical system of an embodiment and a cross-sectional view of an image pickup apparatus;

FIG. 2A is a lens cross-sectional view of an objective optical system of Example 1 of the present disclosure, and FIGS. 2B, 2C, 2D, and 2E are aberration diagrams illustrating spherical aberration (SA), astigmatism (AS), distortion (DT), and chromatic aberration of magnification (CC) of Example 1, respectively;

FIG. 3A is a lens cross-sectional view of an objective optical system of Example 2 of the present disclosure, and FIGS. 3B, 3C, 3D, and 3E are aberration diagrams illustrating spherical aberration (SA), astigmatism (AS), distortion (DT), and chromatic aberration of magnification (CC) of Example 2, respectively;

FIG. 4A is a lens cross-sectional view of an objective optical system of Example 3 of the present disclosure, and FIGS. 4B, 4C, 4D, and 4E are aberration diagrams illustrating spherical aberration (SA), astigmatism (AS), distortion (DT), and chromatic aberration of magnification (CC) of Example 3, respectively;

FIG. 5A is a lens cross-sectional view of an objective optical system of Example 4 of the present disclosure, and FIGS. 5B, 5C, 5D, and 5E are aberration diagrams illustrating spherical aberration (SA), astigmatism (AS), distortion (DT), and chromatic aberration of magnification (CC) of Example 4, respectively;

FIG. 6A is a lens cross-sectional view of an objective optical system of Example 5 of the present disclosure, and FIGS. 6B, 6C, 6D, and 6E are aberration diagrams illustrating spherical aberration (SA), astigmatism (AS), distortion (DT), and chromatic aberration of magnification (CC) of Example 5, respectively;

FIG. 7A is a lens cross-sectional view of an objective optical system of Example 6 of the present disclosure, and FIGS. 7B, 7C, 7D, and 7E are aberration diagrams illustrating spherical aberration (SA), astigmatism (AS), distortion (DT), and chromatic aberration of magnification (CC) of Example 6, respectively;

FIG. 8A is a lens cross-sectional view of an objective optical system of Example 7 of the present disclosure, and FIGS. 8B, 8C, 8D, and 8E are aberration diagrams illustrating spherical aberration (SA), astigmatism (AS), distortion (DT), and chromatic aberration of magnification (CC) of Example 7, respectively;

FIG. 9 is a lens cross-sectional view of an objective optical system for 3D observation of Example 8 of the present disclosure; and

FIG. 10 is a schematic configuration diagram of an endoscope of Example 9 of the present disclosure.

### DETAILED DESCRIPTION

Prior to a description of examples, operation effects of embodiments according to some aspects of the present disclosure will be described. In a specific description of operation effects of the embodiments, specific examples will be described. However, the examples described later as well as the illustrative embodiments are only some of the embodiments encompassed by the present disclosure, and the embodiments include numerous variations. Therefore, the present disclosure is not intended to be limited to the illustrative embodiments.

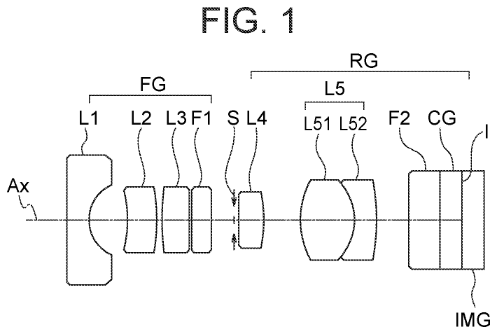

FIG. 1 is a lens cross-sectional view of an objective optical system of an embodiment. The present embodiment is a configuration having one optical axis Ax for 2D observation. Furthermore, the objective optical system has an imager IMG to constitute an image pickup apparatus.

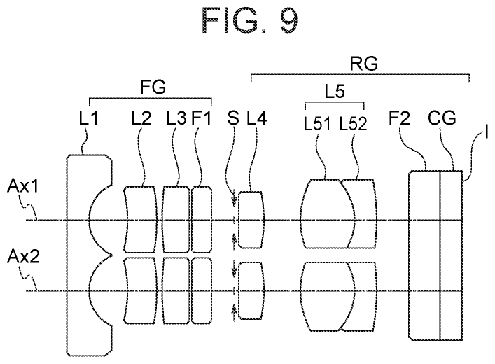

FIG. 9 illustrates a configuration in which two objective optical systems illustrated in FIG. 1 are disposed in parallel. Two objective optical systems are used as an optical system for the right eye and an optical system for the left eye for 3D observation.

The objective optical system of the present embodiment illustrated in FIG. 1 includes a front group FG, an aperture stop S, and a rear group RG in order from the object side. The front group FG includes a first lens L1 having a negative refractive power with a concave surface facing the image side, a second lens L2 having a negative refractive power, and a third lens L3 having a positive refractive power. The rear group RG includes a fourth lens L4 having a positive refractive power and a fifth lens L5 having a positive refractive power. The fifth lens L5 is cemented lenses L51 and L52. The following Conditional Expression (1) is satisfied:

2.3<f3/f5<20 (1)
where
f3 is the focal length of the third lens L3, and
f5 is the focal length of the fifth lens L5.

Furthermore, a filter F1 that is a parallel flat plate is disposed in the front group FG. Furthermore, the filter F2 is cemented to the object side of a cover glass CG of the imager IMG.

It is obvious as a matter of technical common sense that the optical performance realized by the objective optical system is not affected in principle even if a filter that is a parallel flat plate, a lens group, or the like with almost no refractive power (power) is added to the objective optical system of the present embodiment.

The present embodiment is a retrofocus optical system with refractive power arrangement of negative, negative, positive, aperture stop, positive, and positive in order from the object side. Then, in the objective optical system for 2D observation, a wide space is created between the position of an image plane (image pickup surface) I and the fifth lens L5. Thus, it is possible to ensure a stroke for focus adjustment at a time of manufacturing.

Furthermore, in the objective optical system for 3D observation, as illustrated in FIG. 9, two objective optical systems are disposed in parallel, and the fifth lens L5 is moved to perform focus adjustment at the time of manufacturing of the objective optical system. As described later as Example 8, it is possible to ensure a stroke for adjusting the position of the fifth lens L5 illustrated in FIG. 9.

Conditional Expression (1) defines the ratio of the focal length of the third lens L3 to the focal length of the fifth lens L5.

When a value exceeds the upper limit value of Conditional Expression (1), the focal length of the fifth lens L5 becomes smaller. Thus, it is difficult to ensure a stroke of the fifth lens L5 for focus adjustment.

When a value falls below the lower limit value of Conditional Expression (1), the focal length of the third lens L3 becomes smaller relative to the focal length of the fifth lens L5. In the objective optical system for 3D observation, stereoscopic viewing becomes difficult because misalignment due to decentration occurs in the vertical direction (a direction substantially perpendicular to the direction connecting the left eye and the right eye) in an image for the right eye and an image for the left eye. Furthermore, if the focal length of the third lens L3 becomes smaller relative to the focal length of the fifth lens L5, aberration performance such as curvature of field is likely to deteriorate.

In this way, in the present embodiment, it is possible to ensure a stroke of lens movement at a time of focus adjustment in manufacturing of the objective optical system. In addition, in the case of the objective optical system for 3D observation, it is possible to suppress the effects of misalignment or deterioration in aberration performance due to manufacturing errors of the objective optical system for the left eye and the objective optical system for the right eye and to set a short baseline length between the objective optical system for the left eye and the objective optical system for the right eye.

Furthermore, according to a preferred mode of the present embodiment, it is preferable that the fifth lens L5 move along the optical axis Ax for focus adjustment of the objective optical system.

As a result, focus adjustment becomes easy when the objective optical system is manufactured, because the stroke of the fifth lens L5 is ensured.

Furthermore, in a preferred mode of the present embodiment, it is preferable that the following Conditional Expression (2) be satisfied:

0.8<|g2/g1|<2.6 (2)
where
g1 is the focal length of the front group FG, and
g2 is the focal length of the rear group RG.

Conditional Expression (2) defines the ratio of the focal length of the front group FG to the focal length of the rear group RG.

When a value exceeds the upper limit value of Conditional Expression (2), the symmetry between the front group FG and the rear group RG on the front and back of the aperture stop S is lost. As a result, chromatic aberration of magnification and curvature of field become under-corrected.

When a value falls below the lower limit value of Conditional Expression (2), the focal length of the front group FG becomes larger and the beam height in the first lens L1 becomes higher. Thus, it is difficult to set a short baseline length between the objective optical system for the right eye and the objective optical system for the left eye as the 3D objective optical system.

Furthermore, in a preferred mode of the present embodiment, it is preferable that the following Conditional Expression (3) be satisfied:

1.0<|g1/f|<4.0 (3)
where
g1 is the focal length of the front group FG, and
f is the overall focal length of the objective optical system.

Conditional Expression (3) defines the ratio of the focal length of the front group FG to the overall focal length of the objective optical system.

When a value exceeds the upper limit value of Conditional Expression (3), the focal length of the front group FG becomes larger, that is, the refractive power becomes smaller. It is not preferable because consequently, it is impossible to downsize the first lens L1.

When a value falls below the lower limit value of Conditional Expression (3), the focal length of the front group FG becomes smaller, that is, the refractive power becomes larger. As a result, error sensitivity at the time of manufacturing of the front group lens FG is increased. Thus, the angle of view changes when the lens shifts in the optical axis direction of the objective optical system or the direction perpendicular to the optical axis, and a deflection angle is likely to occur. As used herein “deflection angle” refers to the angle between the optical axis and the central axis of the field of view.

Furthermore, in a preferred mode of the present embodiment, it is preferable that the following Conditional Expression (4) be satisfied:

f2/f1<100 (4)
where
f2 is the focal length of the second lens L2, and
f1 is the focal length of the first lens L1.

Conditional Expression (4) defines the ratio between the focal length of the first lens L1 and the focal length of the second lens L2.

When a value exceeds the upper limit value of Conditional Expression (4), the focal length of the first lens L1 becomes smaller. Thus, it is difficult to suppress one-sided blur due to the relative decentration of the first lens L1 and the second lens L2.

Furthermore, in a preferred mode of the present embodiment, it is preferable that the following Conditional Expression (5) be satisfied:

3.0<f4/f<6.0 (5)
where
f4 is the focal length of the fourth lens L4, and
f is the overall focal length of the objective optical system.

Conditional Expression (5) defines the ratio of the focal length of the fourth lens L4 to the overall focal length of the objective optical system.

When a value exceeds the upper limit value of Conditional Expression (5), the focal length of the fourth lens becomes larger, that is, the refractive power becomes smaller. As a result, curvature of field becomes under-corrected.

When a value falls below the lower limit value of Conditional Expression (5), the focal length of the fourth lens L4 becomes smaller, that is, the refractive power becomes larger. As a result, it is difficult to correct chromatic aberration of magnification due to the relative decentration of the fourth lens L4 and the fifth lens L5.

Furthermore, an image pickup apparatus of the present embodiment includes
an objective optical system and an imager IMG disposed on an image plane.

The imager has an image pickup surface and converts an image formed on the image pickup surface by the objective optical system into an electrical signal. The objective optical system is the objective optical system described above.

Thus, it is possible to provide an image pickup apparatus in which a stroke of lens movement at a time of focus adjustment is ensured in manufacturing of the objective optical system, while in the case of the objective optical system for 3D observation, it is possible to suppress the effects of misalignment or deterioration of aberration performance due to manufacturing errors of the objective optical system for the left eye and the objective optical system for the right eye and to set a short baseline length between the objective optical system for the left eye and the objective optical system for the right eye.

Furthermore, an endoscope of the present embodiment includes the image pickup apparatus described above.

Thus, it is possible to provide an endoscope in which a stroke of lens movement at a time of focus adjustment is ensured in manufacturing of the objective optical system, while in the case of the objective optical system for 3D observation, it is possible to suppress the effects of misalignment or deterioration of aberration performance due to manufacturing errors of the objective optical system for the left eye and the objective optical system for the right eye and to set a short baseline length between the objective optical system for the left eye and the objective optical system for the right eye.

Furthermore, in a preferred mode of the present embodiment, it is preferable that the first lens L1 have a plano-concave shape having a negative refractive power with a concave surface facing the image side.

In an endoscope, when mucus or blood inside an organ adheres to the lens surface and interferes with observation, water or air is ejected from an air/water supply nozzle provided on the endoscope distal end to clean the lens surface. In this case, when the object side of the first lens L1 has a convex shape, the cleaning performance tends to deteriorate, and when it has a concave shape, water droplets tend to accumulate in the lens surface. In particular, in the case of a convex shape, the lens surface is easily scratched or cracked when impact is applied to the endoscope distal end at a time of carrying or cleaning the endoscope. By forming the first lens L1 in a plano-concave shape having a negative refractive power with a concave surface facing toward the image side, it is possible to make a configuration in which scratches or cracks are less likely to occur against impact while ensuring cleaning performance and drainage performance for the lens surface.

Furthermore, in a preferred mode of the present embodiment, it is preferable that the second lens L2 have a meniscus shape having a negative refractive power with a convex surface facing the image side.

Thus, it is possible to make a configuration that easily suppresses one-sided blur even when relative decentration of the first lens L1 and the second lens L2 occurs due to manufacturing errors that occur within a tolerance range of the parts such as lens and mirror frame.

Furthermore, in a preferred mode of the present embodiment, in the objective optical system for 3D observation, it is preferable that the first lens L1 be a single lens having two spherical-dome concaves provided with a distance of a baseline length, and the first lens L1 is common to the objective optical system for the left eye and the objective optical system for the right eye.

Thus, it is possible to set a short baseline length while reducing the outer diameter of the distal end of the objective optical system, compared with when different first lenses are disposed in the objective optical system for the left eye and the objective optical system for the right eye. Since the baseline length can be reduced, it is possible to reduce parallax between a left-eye image acquired by the optical system for the left eye and a right-eye image acquired by the optical system for the right eye even in close proximity to a subject, and to suppress eye strain at a time of 3D observation.

Examples will be described below.

### Example 1

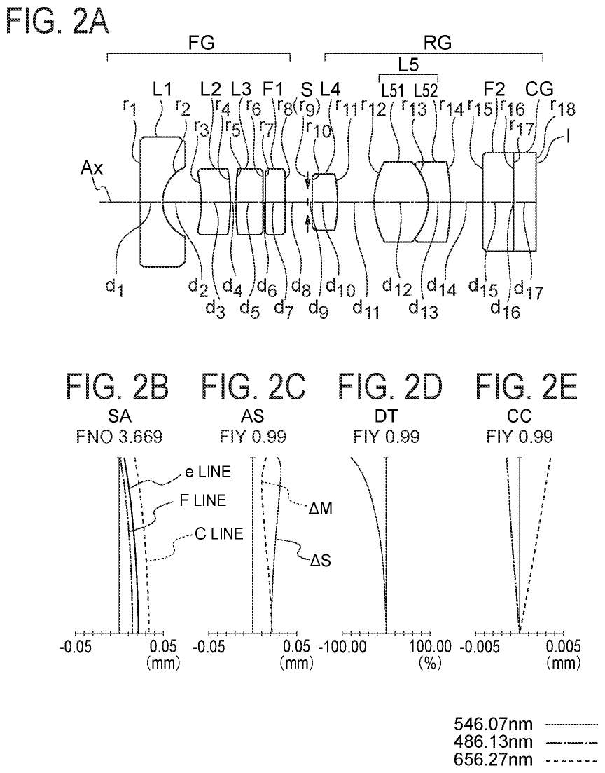

An objective optical system of Example 1 will be described. FIG. 2A is a lens cross-sectional view of the objective optical system of Example 1. The objective optical system includes a front group FG, an aperture stop S, and a rear group RG in order from the object side. The front group FG includes a plano-concave first lens L1 having a negative refractive power with a concave surface facing the image side, a meniscus-shaped second lens L2 having a negative refractive power with a convex surface facing the image side, a plano-convex third lens L3 having a positive refractive power with a convex surface facing the object side, and a filter F1. The rear group RG includes a plano-convex fourth lens L4 having a positive refractive power with a convex surface facing the image side, a biconvex positive lens L51, a meniscus-shaped lens L52 having a negative refractive power with a convex surface facing the image side, a cover glass F2, and a CMOS cover glass CG. The aperture stop S is disposed between the front group FG and the rear group RG. An image plane I exists on the image side of the cover glass CG.

The lens L51 and the lens L52 are cemented to form a fifth lens L5 which is a cemented lens having a positive refractive power.

A wavelength range-limiting coat to limit infrared light may be applied to the surfaces of the filters F1 and F2 that are parallel flat plates. Furthermore, a multilayer film for limiting a wavelength range may be applied to the surface of the cover glass CG. d16 is an adhesive layer.

FIGS. 2B, 2C, 2D, and 2E are aberration diagrams illustrating spherical aberration (SA), astigmatism (AS), distortion (DT), and chromatic aberration of magnification (CC) of Example 1, respectively.

These aberration diagrams are illustrated for the wavelengths of 546.07 nm (e-line), 656.27 nm (C-line) and 486.13 nm (F-line). Furthermore, in each diagram, FNO indicates F number and FIY indicates the maximum image height. This is applicable to the aberration diagrams below. In each aberration diagram, the horizontal axis represents the amount of aberration. For spherical aberration, astigmatism, and aberration of magnification, the unit of the amount of aberration is mm. Furthermore, for distortion, the unit of the amount of aberration is %. Furthermore, the unit of the image height is mm.

### Example 2

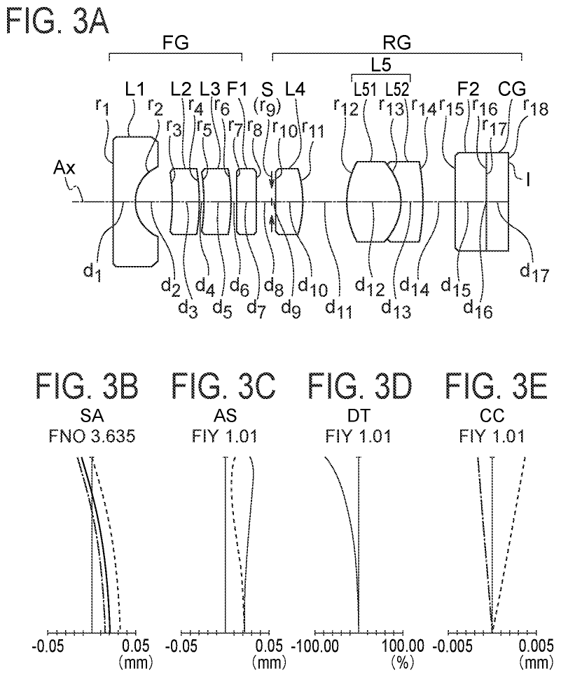

An objective optical system of Example 2 will be described. FIG. 3A is a lens cross-sectional view of the objective optical system of Example 2. The objective optical system includes a front group FG, an aperture stop S, and a rear group RG in order from the object side. The front group FG includes a plano-concave first lens L1 having a negative refractive power with a concave surface facing the image side, a meniscus-shaped second lens L2 having a negative refractive power with a convex surface facing the image side, a meniscus-shaped third lens L3 having a positive refractive power with a convex surface facing the image side, and a filter F1. The rear group RG includes a plano-convex fourth lens L4 having a positive refractive power with a convex surface facing the image side, a biconvex positive lens L51, a meniscus-shaped lens L52 having a negative refractive power with a convex surface facing the image side, a cover glass F2, and a CMOS cover glass CG. The aperture stop S is disposed between the front group FG and the rear group RG. An image plane I exists on the image side of the cover glass CG.

The lens L51 and the lens L52 are cemented to form a fifth lens L5 which is a cemented lens having a positive refractive power.

A wavelength range-limiting coat to limit infrared light may be applied to the surfaces of the filters F1 and F2 that are parallel flat plates. Furthermore, a multilayer film for limiting a wavelength range may be applied to the surface of the cover glass CG. d16 is an adhesive layer.

FIGS. 3B, 3C, 3D, and 3E are aberration diagrams illustrating spherical aberration (SA), astigmatism (AS), distortion (DT), and chromatic aberration of magnification (CC) of Example 2, respectively.

### Example 3

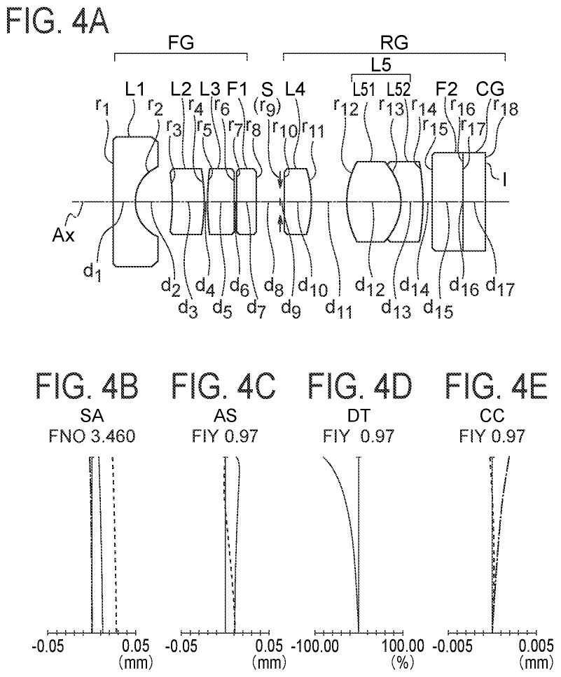

An objective optical system of Example 3 will be described. FIG. 4A is a lens cross-sectional view of the objective optical system of Example 3. The objective optical system includes a front group FG, an aperture stop S, and a rear group RG in order from the object side. The front group FG includes a plano-concave first lens L1 having a negative refractive power with a concave surface facing the image side, a meniscus-shaped second lens L2 having a negative refractive power with a convex surface facing the image side, a plano-convex third lens L3 having a positive refractive power with a convex surface facing the object side, and a filter F1. The rear group RG includes a plano-convex fourth lens L4 having a positive refractive power with a convex surface facing the image side, a biconvex positive lens L51, a meniscus-shaped lens L52 having a negative refractive power with a convex surface facing the image side, a cover glass F2, and a CMOS cover glass CG. The aperture stop S is disposed between the front group FG and the rear group RG. An image plane I exists on the image side of the cover glass CG.

The lens L51 and the lens L52 are cemented to form a fifth lens L5 which is a cemented lens having a positive refractive power.

A wavelength range-limiting coat to limit infrared light may be applied to the surfaces of the filters F1 and F2 that are parallel flat plates. Furthermore, a multilayer film for limiting a wavelength range may be applied to the surface of the cover glass CG. d16 is an adhesive layer.

FIGS. 4B, 4C, 4D, and 4E are aberration diagrams illustrating spherical aberration (SA), astigmatism (AS), distortion (DT), and chromatic aberration of magnification (CC) of Example 3, respectively.

### Example 4

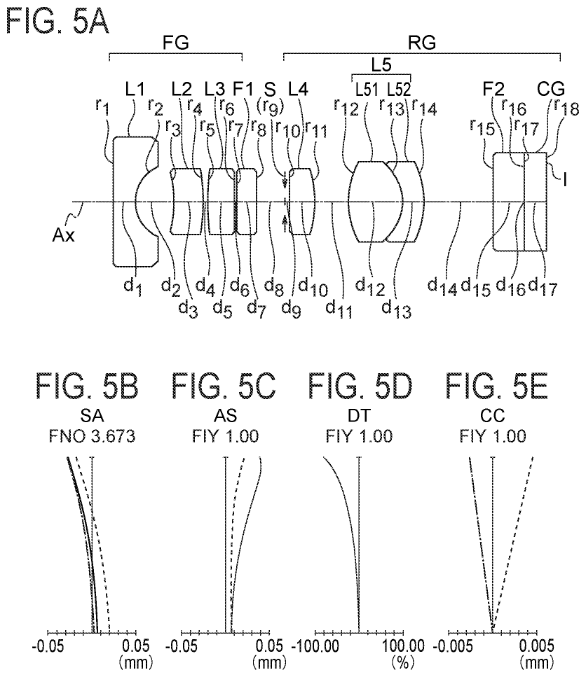

An objective optical system of Example 4 will be described. FIG. 5A is a lens cross-sectional view of the objective optical system of Example 4. The objective optical system includes a front group FG, an aperture stop S, and a rear group RG in order from the object side. The front group FG includes a plano-concave first lens L1 having a negative refractive power with a concave surface facing the image side, a meniscus-shaped second lens L2 having a negative refractive power with a convex surface facing the image side, a plano-convex third lens L3 having a positive refractive power with a convex surface facing the object side, and a filter F1. The rear group RG includes a plano-convex fourth lens L4 having a positive refractive power with a convex surface facing the image side, a biconvex positive lens L51, a meniscus-shaped lens L52 having a negative refractive power with a convex surface facing the image side, a cover glass F2, and a CMOS cover glass CG. The aperture stop S is disposed between the front group FG and the rear group RG. An image plane I exists on the image side of the cover glass CG.

The lens L51 and the lens L52 are cemented to form a fifth lens L5 which is a cemented lens having a positive refractive power.

A wavelength range-limiting coat to limit infrared light may be applied to the surfaces of the filters F1 and F2 that are parallel flat plates. Furthermore, a multilayer film for limiting a wavelength range may be applied to the surface of the cover glass CG. d16 is an adhesive layer.

FIGS. 5B, 5C, 5D, and 5E are aberration diagrams illustrating spherical aberration (SA), astigmatism (AS), distortion (DT), and chromatic aberration of magnification (CC) of Example 4, respectively.

### Example 5

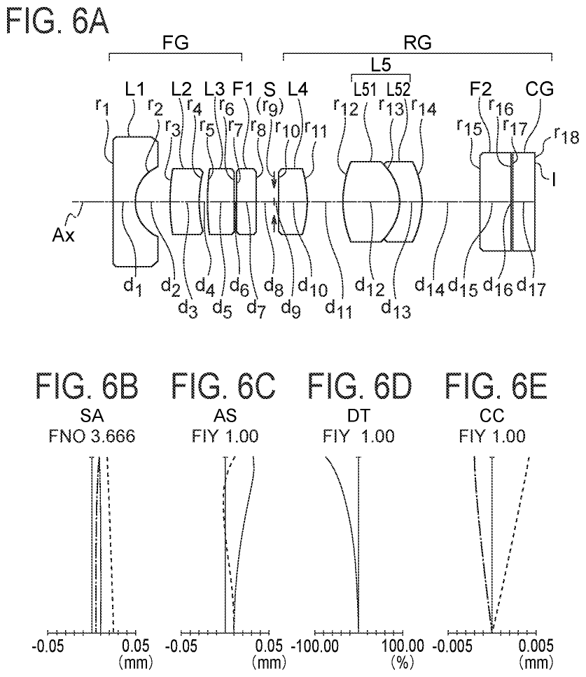

An objective optical system of Example 5 will be described. FIG. 6A is a lens cross-sectional view of the objective optical system of Example 5. The objective optical system includes a front group FG, an aperture stop S, and a rear group RG in order from the object side. The front group FG includes a plano-concave first lens L1 having a negative refractive power with a concave surface facing the image side, a meniscus-shaped second lens L2 having a negative refractive power with a concave surface facing the image side, a plano-convex third lens L3 having a positive refractive power with a convex surface facing the object side, and a filter F1. The rear group RG includes a plano-convex fourth lens L4 having a positive refractive power with a convex surface facing the image side, a biconvex positive lens L51, a meniscus-shaped lens L52 having a negative refractive power with a convex surface facing the image side, a cover glass F2, and a CMOS cover glass CG. The aperture stop S is disposed between the front group FG and the rear group RG. An image plane I exists on the image side of the cover glass CG.

The lens L51 and the lens L52 are cemented to form a fifth lens L5 which is a cemented lens having a positive refractive power.

A wavelength range-limiting coat to limit infrared light may be applied to the surfaces of the filters F1 and F2 that are parallel flat plates. Furthermore, a multilayer film for limiting a wavelength range may be applied to the surface of the cover glass CG. d16 is an adhesive layer.

FIGS. 6B, 6C, 6D, and 6E are aberration diagrams illustrating spherical aberration (SA), astigmatism (AS), distortion (DT), and chromatic aberration of magnification (CC) of Example 5, respectively.

### Example 6

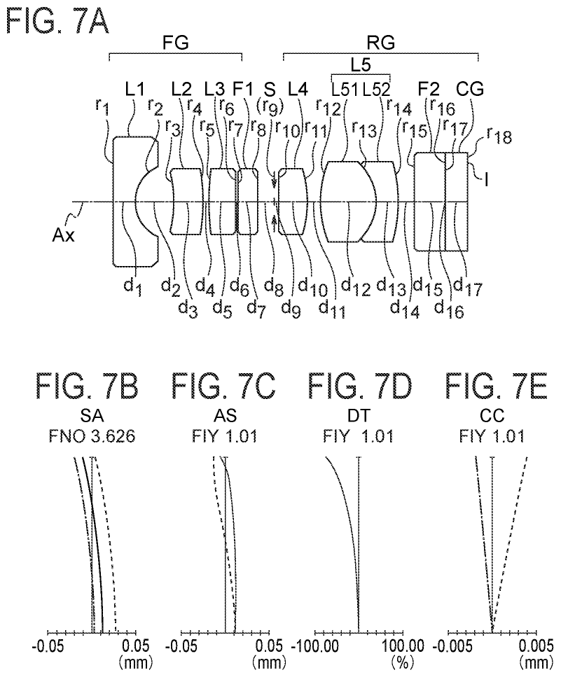

An objective optical system of Example 6 will be described. FIG. 7A is a lens cross-sectional view of the objective optical system of Example 6. The objective optical system includes a front group FG, an aperture stop S, and a rear group RG in order from the object side. The front group FG includes a plano-concave first lens L1 having a negative refractive power with a concave surface facing the image side, a meniscus-shaped second lens L2 having a negative refractive power with a convex surface facing the image side, a plano-convex third lens L3 having a positive refractive power with a convex surface facing the object side, and a filter F1. The rear group RG includes a plano-convex fourth lens L4 having a positive refractive power with a convex surface facing the image side, a biconvex positive lens L51, a meniscus-shaped lens L52 having a negative refractive power with a convex surface facing the image side, a cover glass F2, and a CCD cover glass CG. The aperture stop S is disposed between the front group FG and the rear group RG. An image plane I exists on the image side of the cover glass CG.

The lens L51 and the lens L52 are cemented to form a fifth lens L5 which is a cemented lens having a positive refractive power.

A wavelength range-limiting coat to limit infrared light may be applied to the surfaces of the filters F1 and F2 that are parallel flat plates. Furthermore, a multilayer film for limiting a wavelength range may be applied to the surface of the cover glass CG. d16 is an adhesive layer.

FIGS. 7B, 7C, 7D, and 7E are aberration diagrams illustrating spherical aberration (SA), astigmatism (AS), distortion (DT), and chromatic aberration of magnification (CC) of Example 6, respectively.

### Example 7

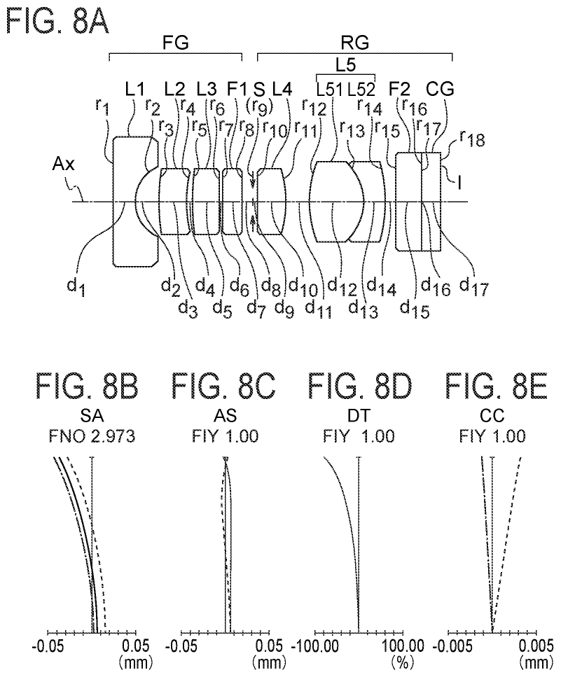

An objective optical system of Example 7 will be described. FIG. 8A is a lens cross-sectional view of the objective optical system of Example 7. The objective optical system includes a front group FG, an aperture stop S, and a rear group RG in order from the object side. The front group FG includes a plano-concave first lens L1 having a negative refractive power with a concave surface facing the image side, a meniscus-shaped second lens L2 having a negative refractive power with a concave surface facing the image side, a plano-convex third lens L3 having a positive refractive power with a convex surface facing the object side, and a filter F1. The rear group RG includes a plano-convex fourth lens L4 having a positive refractive power with a convex surface facing the image side, a biconvex positive lens L51, a meniscus-shaped lens L52 having a negative refractive power with a convex surface facing the image side, a cover glass F2, and a CMOS cover glass CG. The aperture stop S is disposed between the front group FG and the rear group RG. An image plane I exists on the image side of the cover glass CG.

The lens L51 and the lens L52 are cemented to form a fifth lens L5 which is a cemented lens having a positive refractive power.

A wavelength range-limiting coat to limit infrared light may be applied to the surfaces of the filters F1 and F2 that are parallel flat plates. Furthermore, a multilayer film for limiting a wavelength range may be applied to the surface of the cover glass CG. d16 is an adhesive layer.

FIGS. 8B, 8C, 8D, and 8E are aberration diagrams illustrating spherical aberration (SA), astigmatism (AS), distortion (DT), and chromatic aberration of magnification (CC) of Example 7, respectively.

Numerical data of the examples above is listed below. As for the symbols, r is the radius of curvature of each lens surface, d is the distance between lens surfaces, ne is the refractive index of each lens at e-line, νd is the Abbe number of each lens, and Fno is F number. Furthermore, the diaphragm is an aperture stop.

### Numerical Example 1

Surface data

| Surface No. | r | d | ne | vd |
|---|---|---|---|---|
| 1 | ∞ | 0.8097 | 1.88815 | 40.76 |
| 2 | 1.3742 | 1.3649 | | |
| 3 | −6.8061 | 1.0873 | 1.93429 | 18.90 |
| 4 | −9.4365 | 0.1851 | | |
| 5 | 26.3684 | 0.9716 | 1.97189 | 17.47 |
| 6 | ∞ | 0.0694 | | |
| 7 | ∞ | 0.6940 | 1.54170 | 62.47 |
| 8 | ∞ | 0.8791 | | |
| 9 (stop) | ∞ | 0.1157 | | |
| 10 | ∞ | 0.9948 | 1.75453 | 35.33 |
| 11 | −3.8565 | 1.3048 | | |
| 12 | 3.6228 | 1.9433 | 1.69979 | 55.53 |
| 13 | −2.1769 | 0.8097 | 1.97189 | 17.47 |
| 14 | −5.7836 | 1.1451 | | |
| 15 | ∞ | 1.1567 | 1.51825 | 64.14 |
| 16 | ∞ | 0.0231 | 1.51500 | 64.00 |
| 17 | ∞ | 0.8097 | 1.50700 | 63.26 |
| 18 (image plane) | ∞ | | | |

Various data

| | |
|---|---|
| Focal length | 1.00 mm |
| Fno | 3.669 |
| Half angle of view | 79.6° |
| Object point distance | 16.2 mm |
| Image height | 0.992 mm |

### Numerical Example 2

Unit mm
Surface data

| Surface No. | r | d | ne | vd |
|---|---|---|---|---|
| 1 | ∞ | 0.8021 | 1.88815 | 40.76 |
| 2 | 1.3613 | 1.2375 | | |
| 3 | −7.3070 | 1.0313 | 1.93429 | 18.90 |
| 4 | −10.2940 | 0.1833 | | |
| 5 | −7.4311 | 0.9625 | 1.85504 | 23.78 |
| 6 | −7.1919 | 0.1375 | | |
| 7 | ∞ | 0.6875 | 1.54170 | 62.47 |
| 8 | ∞ | 0.6072 | | |
| 9 (stop) | ∞ | 0.1146 | | |
| 10 | ∞ | 0.9854 | 1.75453 | 35.33 |
| 11 | −3.4334 | 1.5790 | | |
| 12 | 4.5059 | 1.8562 | 1.73234 | 54.68 |
| 13 | −2.0173 | 0.8021 | 1.97189 | 17.47 |
| 14 | −5.2300 | 1.1843 | | |
| 15 | ∞ | 1.1459 | 1.51825 | 64.14 |
| 16 | ∞ | 0.0229 | 1.51500 | 64.00 |
| 17 | ∞ | 0.8021 | 1.50700 | 63.26 |
| 18 (image plane) | ∞ | | | |

Various data

| | |
|---|---|
| Focal length | 1.00 mm |
| Fno | 3.635 |
| Half angle of view | 78.4° |
| Object point distance | 16.0 mm |
| Image height | 1.006 mm |

### Numerical Example 3

Unit mm
Surface data

| Surface no | r | d | ne | vd |
|---|---|---|---|---|
| 1 | ∞ | 0.7935 | 1.88815 | 40.76 |
| 2 | 1.3467 | 1.3376 | | |
| 3 | −9.5135 | 1.0655 | 1.93429 | 18.90 |
| 4 | −13.6027 | 0.1814 | | |
| 5 | 11.1267 | 0.9522 | 1.97189 | 17.47 |
| 6 | ∞ | 0.0680 | | |
| 7 | ∞ | 0.6801 | 1.54170 | 62.47 |
| 8 | ∞ | 0.8113 | | |
| 9 (stop) | ∞ | 0.1134 | | |
| 10 | ∞ | 0.9982 | 1.83932 | 37.16 |
| 11 | −3.9644 | 1.2220 | | |
| 12 | 2.4505 | 1.8534 | 1.65425 | 58.55 |
| 13 | −1.9783 | 0.7935 | 1.97189 | 17.47 |
| 14 | −10.7905 | 0.3602 | | |
| 15 | ∞ | 1.1336 | 1.51825 | 64.14 |
| 16 | ∞ | 0.0227 | 1.51500 | 64.00 |
| 17 | ∞ | 0.7935 | 1.50700 | 63.26 |
| 18 (image plane) | ∞ | | | |

Various data

| | |
|---|---|
| Focal length | 1.00 mm |
| Fno | 3.460 |
| Half angle of view | 77.6° |
| Object point distance | 15.9 mm |
| Image height | 0.973 mm |

### Numerical Example 3

Unit mm
Surface data

| Surface no | r | d | ne | vd |
|---|---|---|---|---|
| 1 | ∞ | 0.7968 | 1.88815 | 40.76 |
| 2 | 1.3660 | 1.3433 | | |
| 3 | −3.9859 | 0.9913 | 1.79196 | 47.37 |
| 4 | −11.5793 | 0.1821 | | |
| 5 | 19.6966 | 0.9562 | 1.97189 | 17.47 |
| 6 | ∞ | 0.0683 | | |
| 7 | ∞ | 0.6830 | 1.54170 | 62.47 |
| 8 | ∞ | 1.1179 | | |
| 9 (stop) | ∞ | 0.1138 | | |
| 10 | ∞ | 0.9790 | 1.75453 | 35.33 |
| 11 | −3.7957 | 1.2841 | | |
| 12 | 5.4168 | 1.7905 | 1.59143 | 61.14 |
| 13 | −1.7501 | 0.7968 | 1.97189 | 17.47 |
| 14 | −3.1895 | 2.4289 | | |
| 15 | ∞ | 1.1384 | 1.51825 | 64.14 |
| 16 | ∞ | 0.0228 | 1.51500 | 64.00 |
| 17 | ∞ | 0.7968 | 1.50700 | 63.26 |
| 18 (image plane) | ∞ | | | |

Various data

| | |
|---|---|
| Focal length | 1.00 mm |
| Fno | 3.673 |
| Half angle of view | 78.7° |
| Object point distance | 15.9 mm |
| Image height | 0.999 mm |

### Numerical Example 5

Unit mm
Surface data

| Surface no | r | d | ne | vd |
|---|---|---|---|---|
| 1 | ∞ | 0.7961 | 1.88815 | 40.76 |
| 2 | 1.3647 | 1.1847 | | |
| 3 | 10.4500 | 1.0313 | 1.97189 | 17.47 |
| 4 | 4.0008 | 0.2729 | | |
| 5 | 20.6377 | 0.9771 | 1.93429 | 18.90 |
| 6 | | 0.0682 | | |
| 7 | ∞ | 0.6823 | 1.54170 | 62.47 |
| 8 | ∞ | 0.5231 | | |
| 9 (stop) | ∞ | 0.1137 | | |
| 10 | ∞ | 1.0649 | 1.85504 | 23.78 |
| 11 | −3.7140 | 1.2259 | | |
| 12 | 5.1526 | 1.9365 | 1.77621 | 49.60 |
| 13 | −1.8127 | 0.7961 | 1.97189 | 17.47 |
| 14 | −4.8486 | 1.9366 | | |
| 15 | ∞ | 1.1372 | 1.51825 | 64.14 |
| 16 | ∞ | 0.0227 | 1.51500 | 64.00 |
| 17 | ∞ | 0.7961 | 1.50700 | 63.26 |
| 18 (image plane) | ∞ | | | |

Various data

| | |
|---|---|
| Focal length | 1.00 mm |
| Fno | 3.666 |
| Half angle of view | 77.0° |
| Object point distance | 15.9 mm |
| Image height | 0.998 mm |

### Numerical Example 6

Unit mm
Surface data

| Surface no | r | d | ne | vd |
|---|---|---|---|---|
| 1 | ∞ | 0.8019 | 1.88815 | 40.76 |
| 2 | 1.4664 | 1.3289 | | |
| 3 | −4.8117 | 0.9852 | 1.97189 | 17.47 |
| 4 | −5.4990 | 0.2750 | | |
| 5 | 9.3942 | 0.9843 | 1.93429 | 18.90 |
| 6 | ∞ | 0.0687 | | |
| 7 | ∞ | 0.6874 | 1.54170 | 62.47 |
| 8 | ∞ | 0.5270 | | |
| 9 (stop) | ∞ | 0.1146 | | |
| 10 | ∞ | 1.0728 | 1.77621 | 49.60 |
| 11 | −3.8037 | 0.5233 | | |
| 12 | 4.6841 | 1.9018 | 1.88815 | 40.76 |
| 13 | −2.0621 | 0.8019 | 1.97189 | 17.47 |
| 14 | −9.0955 | 0.5181 | | |
| 15 | ∞ | 1.1456 | 1.51825 | 64.14 |
| 16 | ∞ | 0.0229 | 1.51500 | 64.00 |
| 17 | ∞ | 0.8019 | 1.50700 | 63.26 |
| 18 (image plane) | ∞ | | | |

Various data

| | |
|---|---|
| Focal length | 1.00 mm |
| Fno | 3.626 |
| Half angle of view | 77.0° |
| Object point distance | 16.0 mm |
| Image height | 1.006 mm |

### Numerical Example 7

Unit mm
Surface data

| Surface no | r | d | ne | vd |
|---|---|---|---|---|
| 1 | ∞ | 0.8173 | 1.88815 | 40.76 |
| 2 | 1.3823 | 0.8396 | | |
| 3 | 8.1160 | 1.0041 | 1.93429 | 18.90 |
| 4 | 6.7394 | 0.2335 | | |
| 5 | 23.3503 | 0.9807 | 1.97189 | 17.47 |
| 6 | ∞ | 0.0701 | | |
| 7 | ∞ | 0.7005 | 1.54170 | 62.47 |
| 8 | ∞ | 0.4203 | | |
| 9 (stop) | ∞ | 0.1168 | | |
| 10 | ∞ | 1.0508 | 1.88815 | 40.76 |
| 11 | −2.8453 | 0.7916 | | |
| 12 | 3.3646 | 1.9147 | 1.75844 | 52.32 |
| 13 | −2.0548 | 0.8173 | 1.97189 | 17.47 |
| 14 | −11.2583 | 0.4048 | | |
| 15 | ∞ | 0.8173 | 1.51825 | 64.14 |
| 16 | ∞ | 0.0234 | 1.51500 | 64.00 |
| 17 | ∞ | 0.8173 | 1.50700 | 63.26 |
| 18 (image plane) | ∞ | | | |

Various data

| | |
|---|---|
| Focal data | 1.00 mm |
| Fno | 2.973 |
| Half angle of view | 78.3° |
| Object point distance | 16.3 mm |
| Image height | 1.002 mm |

Conditional values of each of examples are shown below.

Conditional

| expressions | Ex . 1 | Ex .2 | Ex.3 | Ex .4 |
|---|---|---|---|---|
| (1) f3/f5 | 5.97 | 19.50 | 2.43 | 3.97 |
| (2) \|g2/g1\| | 1.72 | 1.88 | 1.24 | 2.47 |
| (3) \|g1/f\| | 1.74 | 1.64 | 2.14 | 1.37 |
| (4) f2/f1 | 21.10 | 21.10 | 25.56 | 5.30 |
| (5) f4/f | 5.12 | 4.55 | 4.72 | 5.03 |

Conditional

| expressions | Ex . 5 | Ex . 6 | Ex . 7 |
|---|---|---|---|
| (1) f3/f5 | 4.93 | 2.38 | 4.97 |
| (2) \|g2/g1\| | 2.52 | 0.82 | 1.37 |
| (3) \|g1/f\| | 1.14 | 3.10 | 1.58 |
| (4) f2/f1 | 4.71 | 81.72 | 42.19 |
| (5) f4/f | 4.34 | 4.90 | 3.20 |

Preferred values for the upper limit value and the lower limit value of each conditional expression are listed below.

It is preferable that the following Conditional Expression (1′) be satisfied instead of Conditional Expression (1).

3.5<f3/f5<15 (1′)

In addition, it is preferable that the following Conditional Expression (1″) be satisfied instead of Conditional Expression (1).

4.9<f3/f5<10 (1″)

It is preferable that the following Conditional Expression (2′) be satisfied instead of Conditional Expression (2).

1.0<|g2/g1|<2.0 (2′)

In addition, it is preferable that the following Conditional Expression (2″) be satisfied instead of Conditional Expression (2).

1.5<|g2/g1|<1.8 (2″)

It is preferable that the following Conditional Expression (3′) be satisfied instead of Conditional Expression (3).

1.2<|g1/f|<3.2 (3′)

In addition, it is preferable that the following Conditional Expression (3″) be satisfied instead of Conditional Expression (3).

1.5<|g1/f|<2.2 (3″)

It is preferable that the following Conditional Expression (4′) be satisfied instead of Conditional Expression (4).

f2/f1<50 (4′)

In addition, it is preferable that the following Conditional Expression (4″) be satisfied instead of Conditional Expression (4).

f2/f1<30 (4″)

It is preferable that the following Conditional Expression (5′) be satisfied instead of Conditional Expression (5).

3.5<f4/f<5.5 (5′)

In addition, it is preferable that the following Conditional Expression (5″) be satisfied instead of Conditional Expression (5).

4.3<f4/f<5.2 (5″T)

The objective optical system described above may simultaneously satisfy a plurality of configurations. Doing so is preferable to obtain a favorable objective optical system. Furthermore, preferable configurations may be combined as desired. Furthermore, in each conditional expression, only the upper limit value or the lower limit value in the numerical range of a more limited conditional expression may be defined.

### Example 8

FIG. 9 is a lens cross-sectional view of an objective optical system for 3D observation of Example 8. For example, the objective optical system of Example 8 is a configuration in which an optical system for the right eye having an optical axis Ax1 and an optical system for the left eye having an optical axis Ax2 are disposed in parallel. The distance between the optical axis Ax1 and the optical axis Ax2 is defined as the baseline length.

Here, what is called D-cut is performed for the fifth lens L5, in which a part of a circular lens is cut in a straight line. Focus adjustment of the objective optical system for 3D observation needs to be performed independently for the optical system for the left eye and the optical system for the right eye. In the present example, focus adjustment of one of the objective optical systems is performed by moving the position of the imager (not illustrated), and focus adjustment of the other objective optical system is performed by moving the fifth lens L5. In the present example, a space is provided on the front and back of the fifth lens L5 for movement of the fifth lens L5.

Furthermore, in the present example, it is possible to ensure a short baseline length between the objective optical system for the left eye and the objective optical system for the right eye by making the first lens L1 common to the optical system for the left eye and the optical system for the right eye and reducing the beam height of the concave surface in the first lens L1. In addition, it is possible to reduce the refractive power of the fifth lens L5 (positive cemented lens) and reduce deterioration of aberration performance at a time of focus adjustment.

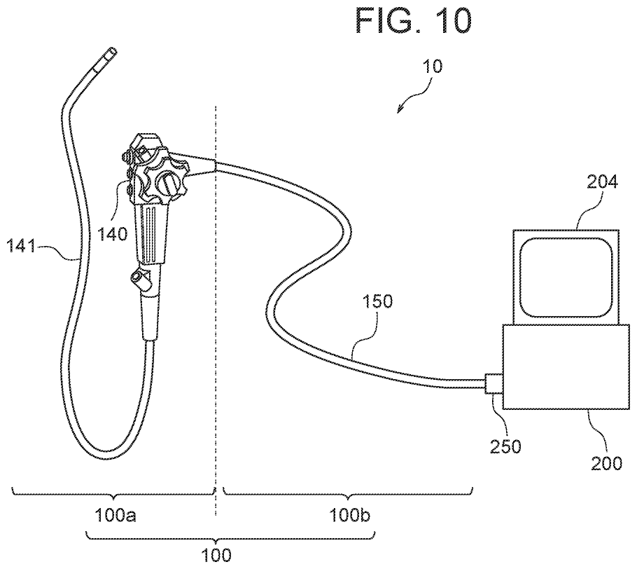

FIG. 10 is a diagram illustrating an endoscope 10 of Example 9. The endoscope 10 is configured with an electronic endoscope 100 and an ex vivo device 200. The electronic endoscope 100 includes a scope section 100a and a connection cord section 100b. Furthermore, the ex vivo device 200 includes a power supply unit, a video processor (not illustrated) that processes a video signal from the electronic endoscope 100, and a display unit 204 that displays a video signal from the video processor on a monitor. The scope section 100a corresponds to an in vivo device.

The scope section 100a is broadly divided into an operating section 140 and an insertion section 141. The insertion section 141 is composed of a flexible member that is elongated and capable of being inserted into the patient's body cavity. It is possible for a user (not illustrated) to perform various operations using, for example, an angle knob provided on the operating section 140.

Furthermore, the connection cord section 100b extends from the operating section 140. The connection cord section 100b includes a universal cord 150. The universal cord 150 is connected to the ex vivo device 200 through a connector 250.

Furthermore, the universal cord 150 communicates a power supply voltage signal, a CMOS drive signal, and the like from the power supply unit and the video processor to the scope section 100a and also communicates a video signal from the scope section 100a to the video processor. It is possible to connect peripheral devices such as a video tape recorder (VTR) deck and a video printer not-illustrated to the video processor in the ex vivo device 200. With the video processor, prescribed signal processing can be applied to a video signal from the scope section 100a and an endoscopic image can be displayed on the display screen of the display unit 204.

The present disclosure is susceptible to various modifications without departing from the spirit thereof. Furthermore, the number of shapes is not necessarily limited to the number depicted in each of the above examples. Furthermore, in each of the above examples, the cover glass is not necessarily disposed. A lens not illustrated in each of the above examples and having substantially no refractive power may be disposed within between lenses or outside between lenses.

As described above, the present disclosure is suitable for an objective optical system, an image pickup apparatus, and an endoscope in which a stroke of lens movement at a time of focus adjustment is ensured, while it is possible to suppress the effects of misalignment or deterioration of optical performance at a time of 3D observation due to manufacturing errors and to set a short baseline length between the objective optical system for the left eye and the objective optical system for the right eye at a time of 3D observation.

According to the present disclosure, it is possible to provide an objective optical system, an image pickup apparatus, and an endoscope in which a stroke of lens movement at a time of focus adjustment is ensured in manufacturing of the objective optical system, while in the case of the objective optical system for 3D observation, it is possible to suppress the effects of misalignment or deterioration of aberration performance due to manufacturing errors of the objective optical system for the left eye and the objective optical system for the right eye and to set a short baseline length between the objective optical system for the left eye and the objective optical system for the right eye at a time of 3D observation.

What is claimed is:

1. An objective optical system comprising: a front group; an aperture stop; and a rear group in order from an object side, wherein
the front group includes a first lens having a negative refractive power with a concave surface facing an image side, a meniscus-shaped second lens having a negative refractive power with a convex surface facing the image side, and a third lens having a positive refractive power,
the rear group includes a fourth lens having a positive refractive power and a fifth lens having a positive refractive power,
the fifth lens is a cemented lens, and
the following Conditional Expression (1) is satisfied:
2.3<f3/f5<20 (1)
where
f3 is a focal length of the third lens, and
f5 is a focal length of the fifth lens.

2. The objective optical system according to claim 1, wherein the fifth lens moves along an optical axis for focus adjustment of the objective optical system.

3. The objective optical system according to claim 1, wherein the following Conditional Expression (2) is satisfied:
0.8<|g2/g1|<2.6 (2)
where
g1 is a focal length of the front group, and
g2 is a focal length of the rear group.

4. The objective optical system according to claim 1, wherein the following Conditional Expression (3) is satisfied:
1.0<|g1/f|<4.0 (3)
where
g1 is a focal length of the front group, and
f is an overall focal length of the objective optical system.

5. The objective optical system according to claim 1, wherein the following Conditional Expression (4) is satisfied:
f2/f1<100 (4)
where
f2 is a focal length of the second lens, and
f1 is a focal length of the first lens.

6. The objective optical system according to claim 1, wherein the following Conditional Expression (5) is satisfied:
3.0<f4/f<6.0 (5)
where
f4 is a focal length of the fourth lens, and
f is an overall focal length of the objective optical system.

7. An image pickup apparatus comprising: an objective optical system; and an imager disposed on an image plane, wherein
the imager has an image pickup surface and converts an image formed on the image pickup surface by the objective optical system into an electrical signal, and
the objective optical system is the objective optical system according to claim 1.

8. An endoscope comprising the image pickup apparatus according to claim 7.
